(19) United States
(12) Patent Application Publication
(10) Pub. No.: US 2006/0286062 A1
(43) Pub. Date: Dec. 21, 2006

(54) Completely natural plant based shampoo and body wash devoid of synthetic ingredients

(76) Inventors: Raymond Albert Schep, Beverly Hills, CA (US); Kort V. Pearson, Laguna Beach, CA (US)

Correspondence Address: RAYMOND A. SCHEP;C/O COLONIAL DAMES, 6820 WATCHER ST., COMMERCE, CA, 90040, US

(21) Appl. No.: 11/445,800

(22) Filed: Jun. 2, 2006

Related U.S. Application Data
(60) Provisional application No. 60/595,102, filed on Jun. 6, 2005.

(51) Int. Cl. A61K 8/97; A61K 36/82; A61K 36/752

(52) U.S. Cl. 424/74; 424/729; 424/736

(57) ABSTRACT

Completely natural hair shampoo or body wash made from all plant and agricultural materials containing anti-oxidants from plant materials, and mild natural surfactants from plant materials. Surfactants, oils, conditioners, preservatives, fragrance and color are all derived from plants and clays. As the surfactants are predominantly from unrefined pant materials, cleansing is mild and non-irritating, and contains a variety of anti-oxidant substances of great benefit to the scalp and skin, and no synthetic ingredients that are foreign to nature and the skin are used.

### REFERENCES CITED [REFERENCED BY]

| | | |
|---|---|---|
| 4568667 | February 1986 | Shirakawa et al. |
| 6007822 | December 1999 | Zhan et al. |
| 6548463 | April 2003 | Myahara et al. |
| 6849096 | February 2005 | Taguchi et al |
| 6869681 | March 2005 | Okamoto et al. |

1. Background of The Invention

The present invention generally relates to the avoidance of synthetic compounds, and their harmful effects, as used for skin and scalp cleansing, and the sole use of materials of natural origin for cleanser, preservative, fragrance, color, conditioning and moisturizing.

2. Description of the prior art.

Much interest in natural cleansers has developed in the consumer and numerous cleansing agents described as natural are being marketed to the public to take advantage of this interest. Investigation into the ingredients and formulation of all cleansers marketed and represented as natural as encountered by the authors reveal that they are conventional cleansers using synthetic detergents such as sodium or ammonium laurel sulfate, or TEA lauryl sulfate, cocoamidopropyl betaine, to which relatively small amounts of plant extracts have been added, the most common being such plants as chamomile, calendula, aloe, and others. The preservatives are synthetic such as DM DM hydantoin, and the isothiazolinones. Conditioners may be pyrrolidinone derived compounds and the fragrances synthetically derived perfumes. Except for the plant extracts of questionable quantity and quality in the cleanser, none of these compounds benefit the skin by contributing a wealth of antioxidants and plant steroids.

Saponin from Soapbark (Quillaja sp) and even Yucca sp does occur in some natural shampoos marketed, but once again the shampoo also contains conventional synthetic preservatives and synthetic fragrances, and usually the main surfactant is still the laurel sulfates. We have found no shampoo or wash marketed today that is formulated solely from plant derived ingredients and natural clay when all ingredients are taken into consideration including the color, conditioners, fragrance and preservative.

In addition conventional surfactants are designed to be extremely effective emulsifiers, stripping the skin and scalp bare of its natural oils and emollients. This however, results in irritation, dry skin and inflammation, opening it up to loss of hair, dandruff and itching and flaking.

### SUMMARY OF THE INVENTION

The object of the invention is to provide a new shampoo, body wash or cleanser that is totally derived form plant materials to provide mild skin friendly cleanser with a wealth of synergistic antioxidants and plant steroids that benefit the skin and scalp and do not promote inflammation, loss of hair, dryness and flaking.

Tea saponin liquids are such as one derived from Camellia Sinensis seeds or any Camellia species that contains healthful tea polyphenols and natural preservatives. Any surfactant made from plant materials may be used as co-surfactants especially those that form a synergic foaming cleansing material such as a vegetable derived glucoside such as coconut oil glucoside. Additionally, powdered Jatropha Cardiophylla plant (Limberbush or Dragonblood) or tea made from the plant also assists in the synergy. Any Jatropha species that has surfactant activity may be used such as J. dioica, J. cinerea, or J.cuneata. These have sap that becomes blood red upon exposure to air. Both tea saponin and J Cardiophylla provide healthful and scalp or skin protecting anti-oxidants to the cleanser. One such anti-oxidant contained in the tea saponin is epigallocatechin gallate with known therapeutic and skin stimulating qualities. Alkyl glucoside is a glucoside made from any source of fatty alcohols that is derived from plant or animal oils or fats. One example is coconut oil derived fatty alcohols known by the international nomenclature cosmetic ingredient name as Coco glucoside and marketed as Plantacare 818 by Cognis. Any thickener from natural origin may be used, such as Locust bean Gum, guar gum, xanthan gum, the preferred thickener is xanthan Gum. Natural clay may also be used as a thickener. Any preservative of natural origin may also be used and the preferred preservative is grapefruit seed extract, also known in the trade as Citricidal. Any vinegars or lemon juice in sufficient quantity to lower the ph blow 4.5 (preferred 4.0) can be used. Vinegars include apple derived or grape derived. Fragrancing can be provided by natural essential oils and additional color can be provided by red or brown clay, blue chamomile oil, or astaxanthin from red algae. Both of aforementioned are powerful anti-oxidant materials. Conditioners and moisturizers are provided by vegetable oils or hydrolyzed protein.

### DETAILED DESCRIPTION OF THE PREFERRED EMBODIMENT

The preferred surfactant is tea saponin and the preferred co-surfactant is either coconut oil glucoside known as coco glucoside or corn oil glucoside known as decyl glucoside and the remaining optional co-surfactant is finely powdered Jatropha dioica root and stem powder or a tea made from the plant. The predominant surfactant is tea saponin and the rest may be present in lesser quantity. The preferred conditioner is either hydrolyzed soy or keratin protein. The preferred moisturizing oils are the oils that contain high amounts of anti-inflammatory omega-3 fatty acids such as canola oil, flaxseed oil hempseed oil, with the latter having top preference. Tocotrienol and astaxanthin may be added to prevent rancidity in these oils. The preferred thickeners are a synergistic combination of a clay marketed as Gelwhite NF and xanthan gum to provide an excellent consistency.

The preferred preservative is organic raw apple cider vinegar. There is no preference on natural essential oils but lavender, orange, bay, wintergreen, spearmint, rosewood, patchouli, vetiver, sandalwood are most used in these formulations. Organic forms of these oils are preferred. For coloring red, brown, or white clay can be used or blue chamomile oil (Guaiazulene) and astaxanthin.

The description of ingredients and how typical formulations are made is as follows:

Example Formula One

| (1) Water | QS to 100.0% |
|---|---|
| (2) Xanthan Gum | 2.0% |
| (3) Montmorillonite clay powder | 3.0% |
| (4) Grapefruit seed extract | 1.0% |
| (5) Coco glucoside | 5.0% |
| (6) Tea Saponin (Camellia Sinensis) | 15.0% |

Example Formula Two

| (1) Water | QS 100.0% |
|---|---|
| (2) Xanthan Gum | 2.0% |
| (3) Montmorillonite clay powder(Gelwhite) | 3.0% |
| (4) Jatropha cardiophylla powder finely milled | 3.0% |
| (5) Grapefruit seed extract | 1.0% |
| (6) Coco glucoside | 4.0% |
| (7) Tea Saponin (Camellia Sinensis seed) | 16.0% |

Any experienced formulator can use percentages of ingredients in varying amounts for greater or less foaming or for a suitable aroma.

The following steps are taken to make the product:
(1) Mix water and xanthan gum until the gum is fully hydrated,
(2) Add gelwhite clay powder to the mixture and mix until fully dispersed.
(3) Add Jatropha powder and mix until fully dispersed.
(4) Add Grapefruit seed extract natural preservative and mix.
(5) Add Coco glucoside and Tea saponin carefully to avoid foaming.

Tea saponin is obtained from a by product of tea seed oil production. The powdered cake from which the oil has been expelled contains 12 to 14% saponins and is treated with hot water to extract the tea saponin.

While the presently preferred embodiments of the present invention have been shown and described, it will be understood that the present invention is not limited thereto, and that various changes and modifications may be made by those skilled in the art without departing from the scope of the invention as set forth in the appended claims.

What is claimed is:

1. A hair cleanser or shampoo or body wash from totally plant derived ingredients containing no synthetic ingredients whatsoever.
Surfactants are selected from plant saponins such as those derived from the seeds of Camellia Sinensis, dried powdered plant material from the roots and stems of Jatropha species, and glucosides made from vegetable oils. The cleanser is rich in plant derived anti-oxidants of known benefit to the scalp or skin and uses a synergic foaming combination of plant glucosides and a plant derived preservative.
Preservatives are chosen from the group of Citricidal or grapefruitseed extract, or vinegar from any plant derived source, or lemon juice or any plant derived acid capable of lowering the pH to below 4.5.
Conditioning agents are animal or vegetable derived hydrolyzed protein such as hydrolyzed soy or keratin or wheat protein.
Additional conditioning agents are vegetable oils or animal derived fats especially those having significant quantities of omega-3 fatty acids. Fragrance are derived from essential oils.
The thickener is derived from xanthan gum, guar gum, locust bean gum or any hydrocellolusic thickening derived from plants or bacteria.
Suitable clays and fine dried plant powder with surfactant property can be used as additional thickening agents.
